(19) United States
(12) Patent Application Publication
(10) Pub. No.: US 2007/0128610 A1
(43) Pub. Date: Jun. 7, 2007

(54) Sample preparation method and apparatus for nucleic acid sequencing

(76) Inventor: Philip R. Buzby, Brockton, MA (US)

Correspondence Address: PROSKAUER ROSE LLP, ONE INTERNATIONAL PLACE 14TH FL, BOSTON, MA, 02110, US

(21) Appl. No.: 11/292,528

(22) Filed: Dec. 2, 2005

(51) Int. Cl. C12Q 1/68; C12M 3/00

(52) U.S. Cl. 435/6; 435/287.2

(57) ABSTRACT

The invention provides methods and apparatus for preparation and sequencing of nucleic acids.

### FIELD OF THE INVENTION

The invention relates generally to methods and apparatus for sequencing nucleic acids obtained from a relatively small population of cells.

### BACKGROUND OF THE INVENTION

Traditional methods for sequencing nucleic acids typically utilize a pool of nucleic acids obtained from a large population of cells. The cell population used to obtain nucleic acids is presumed to be in a uniform biological state because the cells are obtained from the same source. Nucleic acids are extracted from these samples, resulting in a mixture of nucleic acids from the different cells in the sample. Typically, nucleic acids are amplified prior to sequencing.

Amplification of nucleic acids prior to sequencing can result in the inability to detect sequences that are rare or infrequent in the sample form which the nucleic acids were extracted. Limitations of traditional sequencing methods include the inability to analyze nucleic acid samples such that nucleic acids from one or a few cells can be sequenced without losing cell specific information while maintaining an accurate, unbiased nucleic acid population.

There is a need, therefore, for techniques that allow analysis of nucleic acids from a relatively small sample of cells, in particular, techniques that can be used to analyze the genetic content of one or a few cells without having to amplify the nucleic acids prior to its analysis.

### SUMMARY OF THE INVENTION

The present invention is directed to methods and apparatus for nucleic acid sample preparation for sequencing. According to the invention, nucleic acid sample preparation is completed in situ in a sequencing flow cell. Accordingly, in a preferred embodiment, cells are introduced into a microfluidic flow cell where they are lysed, resulting in deposition of cellular nucleic acids on a surface for sequencing. Because sample preparation is done at or near the sequencing surfaces, methods of the invention decrease the potential for loss of nucleic acid material in traditional sample preparation and handling. Methods of the invention also are amenable to automation.

An exemplary flow cell apparatus is shown in FIG. 2. Whole cell preparations are introduced into the flow cell via an inlet. Cell lysis is accomplished by any acceptable method and can occur prior to or at the point of introduction of the sample to the sequencing surface. Once cell lysis is accomplished, nucleic acids are capture by sequence-specific primers bound to the sequencing surface in the flow cell. Unbound material is rinsed and sequencing proceeds as described below.

Cells can be lysed by exposing the cells to a detergent. In other embodiments, the cells can be exposed to shearing or hypotonic conditions, or they can be sonicated.

In a preferred embodiment, at least a portion of the nucleic acids prepared according to the invention are individually optically resolvable. Nucleic acids can be attached directly to the surface, for example via direct amine coupling to an epoxide group. In another embodiment, the released nucleic acids are immobilized via a binding pair. For example, released nucleic acids can contain or can be modified to contain a member of a binding pair and the surface can be coated with the other member of the binding pair. The binding pair can be, for example, antibody/antigen, biotin/streptavidin, or receptor/ligand. In still another embodiment, the nucleic acids can be immobilized by hybridizing to a primer that is attached to the surface. The primer can be attached to the surface via an epoxide group or a binding pair. In still another embodiment, both the nucleic acid to be sequenced and the primer can be attached to the surface.

In one preferred embodiment, the surface comprises an epoxide coating that has been passivated to prevent non-specific binding. The surface can be passivated (e.g., blocked) with any suitable passivating (e.g., blocking) agent. In one embodiment, the epoxide coated surface is passivated with phosphate.

Random primers may be attached to the surface for nucleic acid capture. Alternatively, nucleic acids may be modified for hybridization to support-bound primers of known sequence. For example, isolated nucleic acids may be tailed with a sequence that is complementary to a portion of a surface-bound primer. Nucleic acids may be added by ligation or enzymatically by, for example, terminal transferase addition. In a preferred embodiment, isolated sample nucleic acids are polyadenylated and hybridized to poly-d(T) primers on the sequencing surface.

Released nucleic acids can be fragmented such that the resulting fragments are suitable for immobilization and/or analysis. Nucleic acids can be fragmented, for example, by sonication or by digesting the nucleic acids with a suitable enzyme. Suitable enzymes include endonucleases such as restriction endonucleases. In one embodiment, the lysis buffer includes the enzyme.

Prior to releasing the nucleic acids, the cell or cells can optionally be captured or immobilized onto a surface of the flow cell. To immobilize the cells, a surface of the flow cell can be coated with a member of a binding pair. The surface of the cells can either include the corresponding member of the binding pair or the cells can be labeled with the corresponding member of the binding pair. The cells and nucleic acids can be immobilized on the same surface or on different surfaces of the flow cell.

To monitor cells, the cells can be labeled with a detectable marker. For example, the cells can be labeled with a fluorescent dye. Immobilized labeled cells can be detected using standard light microscopy or by detecting the fluorescent label. The detectable marker can be present, for example on biotin used to coat the cells, or on an antibody used to label the cells, such as an antibody that recognizes a surface marker or a lectin that recognizes a cell surface carbohydrate. The surface of the flow cell can be scanned to detect the presence of the capture cells.

The cells can be any suitable sample obtained from an animal, plant, bacterium, fungus, or any other cellular organism. The cells may be obtained directly from an organism or from a biological sample obtained from an organism, e.g., from blood, urine, cerebrospinal fluid, seminal fluid, saliva, sputum, stool and tissue. Any tissue or body fluid specimen may be used as a source of cells for use in the invention. Cultured cells may also be used, such as a primary cell culture, a cell line, bacterial culture and the like. The cells can be infected with a virus or other intracellular pathogen.

A sub-population of cells can be isolated from the sample and introduced into the flow cell. A sub-population of cells can be isolated, for example, by fluorescence activated cell sorting (FACS) or by laser assisted micro-dissection.

The present invention also includes apparatus suitable for preparation and sequencing of nucleic acids. In one embodiment, the apparatus comprises a flow cell having an inlet, an outlet, and a surface treated to allow nucleic acids to be attached thereto. The apparatus optionally includes nucleic acids or primers attached to a surface of the flow cell. The nucleic acids or primers can be attached to the surface such that at least a portion of the nucleic acids or primers are individually optically resolvable.

The apparatus optionally can include a second surface. One surface of the flow cell can be treated to allow nucleic acids to be attached thereto and the second surface of the flow cell can be treated to allow the immobilization of cells thereto. Preferably, the two surfaces can be in fluid communication with each other. The surfaces can be in the same region or chamber of the flow cell or can be in different regions or chambers of the flow cell. In one embodiment, the two surfaces are in different chambers that are connected by a valve. The valve can be opened to allow fluid communication or closed to prevent fluid communication between the two chambers.

The apparatus optionally includes a microscope, wherein the flow cell is operably positioned on the microscope stage such that the added nucleotides can be identified using the microscope. In one embodiment, the microscope uses a total internal reflection objective.

### BRIEF DESCRIPTION OF THE DRAWINGS

FIG. 1 shows an exemplary imaging system of the present invention.

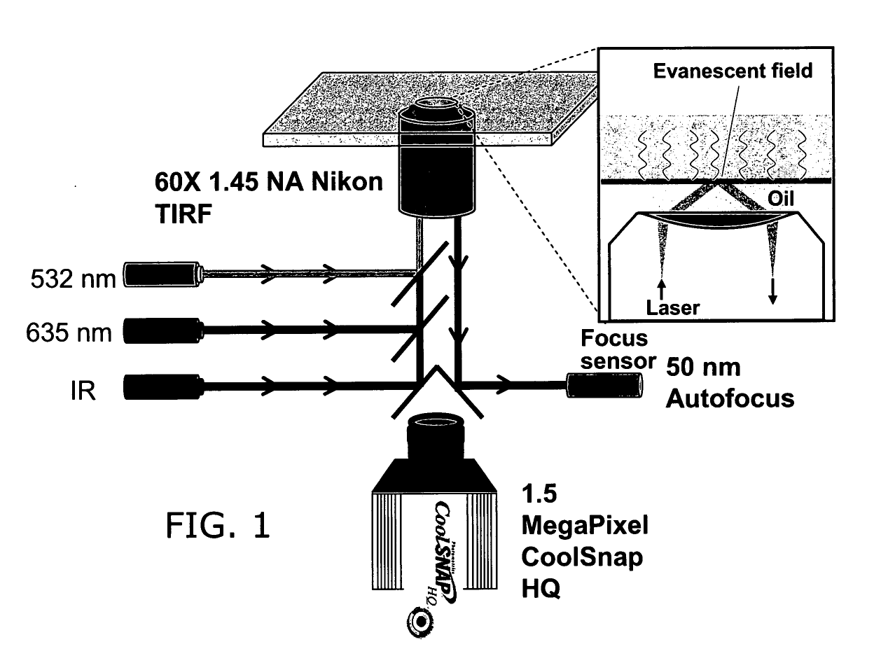

FIG. 2 shows an exemplary flow cell.

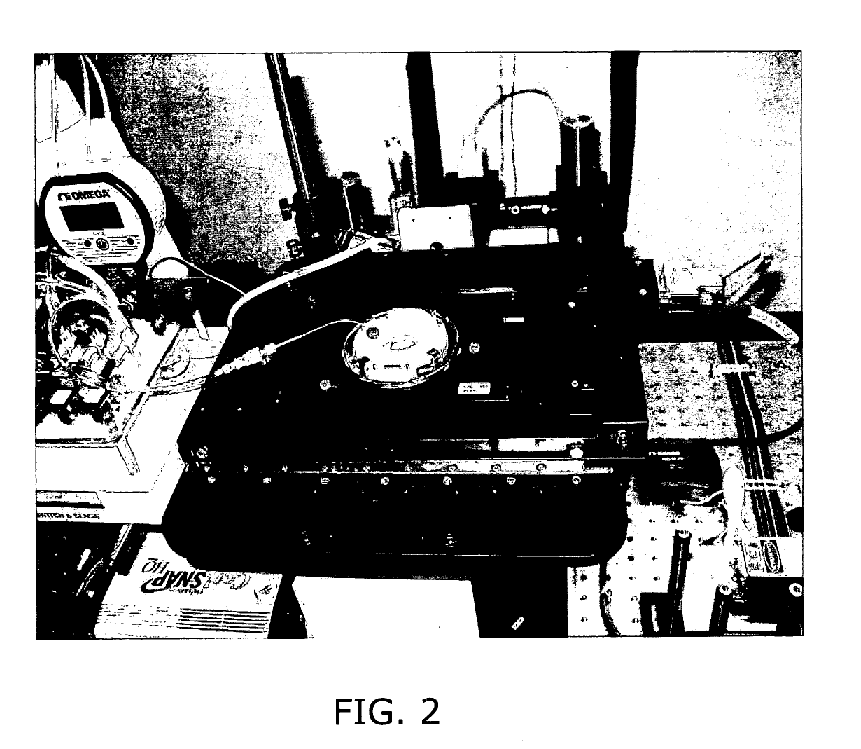

FIG. 3 shows a structure of Cy5 attached to the four common nucleotides.

### DETAILED DESCRIPTION OF THE INVENTION

The present invention is directed to methods and apparatus for nucleic acid sample preparation directly on a sequencing surface. According to the invention, a single cell, or a group of cells, are introduced into a sequencing apparatus in which surface-bound duplex is sequenced by template-dependent synthesis. In a preferred embodiment, a cell or cells is/are introduced into a microfluidic flow cell and processed to isolate nucleic acid from the cells. Nucleic acid extracted from the cells is preferably fragmented to a reasonable size (preferably from about 20 nucleotides in length to about 2000 nucleotides in length) and hybridized to support-bound primers. The surface is rinsed to remove cellular material, unbound nucleic acid and the like, and template-dependent sequencing-by-synthesis is conducted on the support-bound duplex.

Cells for use in the invention can be obtained, for example, from any animal tissue. The tissue sample can be disrupted, for example, by mechanical or enzymatic means to yield a cell suspension. The animal tissue may be any adult or embryonic tissue derived, for example from pancreas, liver, smooth muscle, striated muscle, cardiac muscle, bone, bone marrow, cartilage, spleen, thymus, tonsil, Peyer's patch, lymph nodes, thyroid, epidermis, dermis, subcutaneous, heart, lung, vasculature, endothelium, blood cells, bladder, kidney, esophagus, stomach, small intestine, large intestine, adipose, reproductive tract, eye, lung, connective, endocrine, mesentery, and umbilical tissue.

In general, cells for use in methods and apparatus can be prepared from a tissue sample using any suitable method, such as by gently teasing apart the excised tissue in a suitable buffer and/or by digestion of excised tissue with a suitable enzyme. Suitable enzymes include trypsin and collagenase (for example, collagenase A). The tissue can be, for example, perfused with or incubated in an enzyme-containing buffer of suitable pH and tonic strength to allow cells to be released from the tissue. Debris and any remaining particles of tissue can be removed, to form a cell suspension. The cell suspension may be concentrated using suitable methods, such as centrifugation or diafiltration.

Cell lines can also be used in methods and apparatus of the present invention. Cell lines include, but are not limited to, those available from cell repositories such as the American Type Culture Collection (on the world wide web at atcc.org), the World Data Center on Microorganisms (on the world wide web at wdcm.nig.acjp), European Collection of Animal Cell Culture (on the world wide web at ecacc.org) and the Japanese Cancer Research Resources Bank (on the world wide web at cellbank.nihs.go.jp). These cell lines include, but are not limited to 293, CHO, MCF7, LNCap, T-5, BSC-1, BHK-21, Phinx-A, 3T3, HeLa, PC3, DU145, ZR 75-1, HS 578-T, DBT, Bos, CV1, L-2, RK13, HTTA, HepG2, BHK-Jurkat, Daudi, RAMOS, KG-1, K562, U937, HSB-2, HL-60, MDAHB231, C2C12, HTB-26, HTB-129, HPIC5, A-431, CRL-1573, 3T3L1, Cama-1, J774A.1, HeLa 229, PT-67, Cos7, OST7, HeLa-S, THP-1, and NXA. Additional cell lines for use in the methods and apparatus of the present invention can be obtained, for example, from cell line providers such as Clonetics Corporation (Walkersville, Md.; on the World Wide Web at clonetics.com).

A sub-population of cells can be isolated from the sample and introduced into the flow cell. A sub-population of cells can be isolated from the sample, for example, by fluorescence activated cell sorting (FACS) or by laser assisted micro dissection. Suitable cell surface markers can be used to select and isolate a sub-population of cells. Cells can be incubated, for example, with fluorescently labeled antibodies that recognize a particular cell surface marker known to be on a cell type of interest. Cells labeled with the antibody can be subjected to FACS or laser assisted micro-dissection. One of skill will recognize that there are numerous cell surface markers that can be used to isolate a sub-population of cells for use in the methods and apparatus of the invention. These cell surface markers include, but are not limited to carbohydrates, proteins, glycoproteins, MHC complexes, and receptor proteins. For example, to isolate a sub-population of cells corresponding to immune cells, one or more leukocyte differentiation antigens can be used. For example, as shown in Table I, the indicated surface antigen can be used as a cell surface marker to isolate the indicated cell type.

TABLE I

| Surface Antigen | Cell Type |
|---|---|
| CD2 | T lymphocytes |
| CD4 | T cell subset |
| CD5 | T lymphocytes |
| CD6 | T lymphocytes |
| CD8 | T cell subset |
| CD27 | Naïve CD4 T cell subset |
| CD31 | Naïve CD4 T cell subset |
| CD25 | Activated T cells |
| CD69 | Activated T cells |
| HLA-DR | Activated T cells, APC |
| CD28 | T lymphocytes |
| CD152 (CTLA-4) | Activated T cells |
| CD154 (CD40L) | Activated T cells |
| CD19 | B lymphocytes |
| CD20 | B lymphocytes |
| CD21 | B lymphocytes |
| CD40 | Antigen presenting cells |
| CD134 (OX40) | Antigen presenting cells |
| By-1 and 2 | Antigen presenting cells |
| CD45 | Leukocytes |
| CD83 | Mature dendritic cells |
| CMRF-44 | Mature dendritic cells |
| CMRF-56 | Mature dendritic cells |
| OX40L | Dendritic cells |
| DEC-205 | Dendritic cells |
| TRANCE/RANK receptor | Dendritic cells |

Other suitable surface markers include receptor proteins, such as growth factor receptor proteins. Suitable growth factor receptors are well known in the art and include, but are not limited to receptors for platelet-derived growth factor (PDGF), epidermal growth factor (EGF), insulin-like growth factor (IGF), transforming growth factor β (TGF-β), fibroblast growth factor (FGF), interleukin 2 (IL-2), nerve growth factor (NGF), interleukin 3 (IL-3), interleukin 4 (IL-4), interleukin 1 (IL-1), interleukin 6 (IL-6), interleukin 7 (IL-7), granulocyte/macrophage colony-stimulating factor (GM-CSF), erythropoietin and the like. One of skill in the art recognizes that the term growth factor as used herein generally includes cytokines and colony stimulating factors. In addition, tumor cell surface antigens can be used to isolate tumor cells from a sample for use in the methods and apparatus of the present invention. Suitable tumor cell surface antigens are well known in the art and include, but are not limited to the antigens listed in Table II.

TABLE II
Tumor cell surface antigen

| Antigen(s) | Tumor Cell Type |
|---|---|
| CEA | Colorectal, thyroid carcinoma, others |
| Her2/neu | Breast, ovarian carcinomas |
| CM-1 | Breast |
| MUC-1 | Pancreatic carcinoma, others |
| 28K29 | Lung adenocarcinoma, large cell carcinoma |
| E48 | Head and neck squamous cell carcinoma |
| U36 | Head and neck squamous cell carcinoma |
| NY-ESO-1 | Esophageal carcinoma, melanoma, others |
| KU-BL 1-5 | Bladder carcinoma |
| NY CO 1-48 | Colon carcinoma |
| HOM MEL 40 | Melanoma |
| OV569 | Ovarian carcinoma |
| ChCE7 | Neuroblastoma, renal cell carcinoma |
| CA19-9 | Colon carcinoma |
| CA125 | Ovarian carcinoma |
| Gangliosides | melanoma, neuroblastoma, others |
| (GM2, GD2, | |
| 9-o-acetyl-GD3, GD3) | |

After introducing the cells into the flow cell, the nucleic acids are released. Nucleic acids can be released, for example, by lysing the cells. Cells can be lysed by exposing the cells to a detergent containing solution. Generally, nucleic acids can be released from cells by a variety of techniques such as those described by Maniatis, et al., Molecular Cloning: A Laboratory Manual, Cold Spring Harbor, N.Y., pp. 280-281 (1982). Nucleic acids can be released from cells as described in U.S. Patent Application 2002/0190663 A1, published Oct. 9, 2003, the teachings of which are incorporated herein by reference in their entirety. In a preferred embodiment, nucleic acids are released using conditions or lysis buffers that do not include toxic organic solvents. The extraction methods used should be capable of releasing nucleic acids from a relatively small quantity of cells, such as 1 to about 10⁶ cells or about 10⁵, 10⁴, 10³ 10², or 10 cells. Commercially available kits or reagents can be used to release the nucleic acids, such as the ArrayPure™ Nano-scale RNA purification Kit from Epicentre (on the World Wide Web at epicentre.com). The RNA purification kit can be modified to use RNase in place of DNase, to prepare RNA-free DNA.

In one embodiment, the cells are exposed to a suitable buffer containing a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulfonate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid. Preferred buffer concentration is from about 5 mM to about 500 mM in solution or in solution with the sample. The buffer concentration in the lysing solution can be between about 10 mM and 300 mM.

In other embodiments, the cells can be exposed to shearing or hypotonic conditions, or can be sonicated. Cells can also be subjected to photolysis as described, for example, on page 1541 of He, et al., Anal. Chem. 77:1539-1544 (2005), the teachings of which are incorporated herein by reference.

Released nucleic acids are immobilized on a surface of the flow cell. A preferred surface is an epoxide coated glass or fused silica slide or coverslip. However, any surface that has low native fluorescence is useful in the invention. Other surfaces include, but are not limited to, Teflon, polyelectrolyte multilayers, and others. The only requirement of a surface for use in the invention is that it has low native fluorescence and has the ability to bind nucleic acids, either directly or indirectly.

In a preferred embodiment, nucleic acids are attached to a substrate (also referred to herein as a surface) and subjected to analysis by single molecule sequencing. Nucleic acids are released from the cells and flowed into the region of the flow cell that contains the surface to which nucleic acids will be immobilized or are released in the region of the flow cell that contains said surface. Nucleic acids are attached to the surface such that at least a portion of them are individually optically resolvable. After incubating for a sufficient time to allow immobilization of the nucleic acids, unbound nucleic acids can be removed from the region containing the surface. Unbound nucleic acids can be removed, for example, by flowing fresh buffer that does not contain nucleic acids into the region of the flow cell that contains the surface. In addition, the surface can be washed to remove unbound nucleic acids and other cellular components by flushing the surface with a suitable buffer.

Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair nucleic acid binding.

Various methods can be used to anchor or immobilize the nucleic acids to the surface. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the nucleic acid molecules or the primers to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

In one preferred embodiment, the surface comprises an epoxide coating that has been passivated to prevent non-specific binding. The surface can be passivated (blocked) with any suitable passivating (blocking) agent. In one embodiment, the epoxide coated surface is passivated with phosphate.

In still another embodiment, the nucleic acids can contain or be modified to contain primer complementary sequence. For example, nucleotides can be added to the 3′ end of the nucleic acids. The primer can contain sequence that is complementary to the added sequence. In one embodiment, a predetermined number of nucleotides is added to the 3′ end of the nucleic acids. The predetermined number of nucleotides can be added, for example by ligating an oligonucleotide comprising the predetermined number of nucleotides to the nucleic acids. The primers can be attached to the surface of the flow cell and the released nucleic acids containing primer complementary sequence can be immobilized on the surface by incubating the nucleic acids in the presence of the primer-coated surface under conditions suitable for the nucleic acids to hybridize to the attached primers. The primer sequence can be about 10 to about 50 nucleotides in length. The primer sequence and complementary target nucleic acid molecule sequence can be of the same length or of different lengths.

Generally, nucleic acid molecules can be from about 5 bases to about 200 kb in length or can be fragmented such that they are from about 5 bases to about 20 kb in length. The released nucleic acids can be fragmented prior to or after immobilization onto the surface to produce suitable fragments for analysis. Nucleic acids can be fragmented by any method suitable to produce fragmented nucleic acids for analysis. Nucleic acids can be fragmented, for example, by sonication or by digesting the nucleic acid with an enzyme. Suitable enzymes include endonucleases such as restriction endonucleases. In one embodiment, the lysis buffer includes the enzyme. The nucleic acids or fragments thereof can be about 10 kb, about 5 kb, about 1 kb, about 500 bases, about 400 bases, about 300 bases, about 200 bases, about 100 bases, about 50 bases, about 10 bases, in length, or can be any range of lengths therein. The nucleic acids can comprise molecules of different lengths. The nucleic acid molecules may be single-stranded, double-stranded, or double-stranded with single-stranded regions (for example, having stem- and loop-structures).

Prior to releasing the nucleic acids, the cells can be captured or immobilized onto a surface of the flow cell. To immobilize the cells, a surface of the flow cell can be coated with a member of a binding pair. The surface of the cells can either include the corresponding member of the binding pair or the cells can be labeled with the corresponding member of the binding pair. Binding pairs for immobilizing cells of interest can be non-specific, such as biotin/streptavidin. The cells can be labeled with biotin and the surface of the flow cell can be labeled with streptavidin. Biotin labeled cells will be immobilized on the avidin-coated surface and the non-cellular material can be washed away. In another embodiment, a sub-population of cells may be immobilized, for example, by coating a surface of the flow cell with an antibody that recognizes a surface marker present on cells of interest. Cells having the surface marker will be immobilized by binding to the antibody. Suitable cell surface markers are described above. Alternatively, the surface of the flow cell can be coated with a lectin that binds to a carbohydrate present on the surface of a cell of interest. Cells having the carbohydrate on their surfaces will be immobilized by binding to the lectin. After immobilizing the cells onto the surface of the flow cell, unbound cells can be removed from the flow cell, for example, by flushing the flow cell with a suitable buffer.

Alternatively, the cells can be coated with the corresponding member of the binding pair prior to introducing the cells into the flow cell. For example, where the surface of the flow cell is coated with streptavidin, the cells can be labeled with biotinylated antibody, where the antibody recognizes a particular surface marker of the cell. The cells labeled with biotinylated antibody will be immobilized by the binding between the biotin and the streptavidin present on the flow cell surface.

To monitor the capture of cells, the cells can be labeled with a detectable marker. For example, the cells can be labeled with a fluorescent dye and immobilized labeled cells can be detected using standard light microscopy or by detecting the fluorescent label. The detectable marker can be present, for example on biotin used to coat the cells, or on an antibody that recognizes a surface marker or a lectin that recognizes a cell surface carbohydrate. The surface of the flow cell can be scanned to detect the presence of the capture cells.

The immobilized nucleic acids are sequenced. Sequencing comprises exposing the template/primer duplexes to polymerase and at least one nucleotide species under conditions suitable for template-dependent nucleotide addition to the primer. In one embodiment, nucleic acids are hybridized to primers attached to a surface of the flow cell, thereby immobilizing the nucleic acids and forming template/primer duplexes. In an alternative embodiment, the template/primer duplexes are formed by exposing the attached nucleic acids to primers under conditions suitable for forming a template/primer duplexes. The attached nucleic acids can be exposed to primers by flowing a solution containing primers into the flow cell.

The primers can comprise a sequence of any length suitable for hybridizing to the nucleic acid molecules. In one embodiment, the primer comprises a homopolymeric nucleotide sequence, and the nucleic acid molecules contain or have been modified to contain a complementary homopolymeric sequence of the same or different length.

Conditions for hybridizing primers to nucleic acid targets are well known. The annealing reaction is performed under conditions that are stringent enough to guarantee sequence specificity, yet sufficiently permissive to allow formation of stable hybrids at an acceptable rate. The temperature and length of time required for primer annealing depend upon several factors including the base composition, length and concentration of the primer, and the nature of the solvent used, e.g., the concentration of cosolvents such as DMSO (dimethylsulfoxide), formamide, or glycerol, and counterions such as magnesium. Typically, hybridization (annealing) between primers and target nucleic acids is carried out at a temperature that is approximately 5 to 10° C. below the melting temperature of the target-primer hybrid in the annealing solvent. Typically, the annealing temperature is in the range of 55 to 75° C. and the primer concentration is approximately 0.2 μM. Under such conditions, the annealing reaction is usually complete within a few seconds.

As described herein, nucleic acid molecules are analyzed using sequencing-by-synthesis techniques. Nucleic acid molecules are hybridized to a primer to form template/primer complexes on a surface of the flow cell. As described above, the nucleic acid molecule, the primer, or both the nucleic acid molecule and the primer are attached to the surface. Thereafter, primer extension is conducted to identify at least one nucleotide of the template using a nucleotide polymerizing enzyme and a nucleotide (e.g., dATP, dTTP, dUTP, dCTP and/or a dGTP) or nucleotide analog. Where single molecule sequencing is conducted, incorporation of a nucleotide is detected at discrete locations on the surface. Template/primer complexes, as well as the incorporated nucleotides, are individually resolvable in single molecule embodiments. Alternatively, bulk signal from mixed nucleic acid populations or clonal populations of templates, are obtained.

Sequencing can be conducted by introducing a polymerase and at least one nucleotide species comprising an optically-detectable label into the flow cell, under conditions sufficient for template-dependent nucleotide addition to the primer. As used herein nucleotide or nucleotide species includes nucleotide analogs. The unincorporated nucleotide is optionally removed and the nucleotide species incorporated into said primer is identified be detecting the optically-detectable label. Sequencing, as used herein can be performed such that one or more nucleotides are identified in one or more nucleic molecules. Methods according to the invention also include the step of compiling a sequence of the molecule (nucleic acid) based upon sequential incorporation of the extension bases into the primer.

Nucleic acid molecules can be sequenced using single molecule sequencing as described, for example, in U.S. patent application Ser. No. 11/137,928, filed May 25, 2005, U.S. patent application Ser. No. 11/067,102, filed Feb. 25, 2005, and/or and described in U.S. Pat. No. 6,780,591, the teachings of which are incorporated herein in their entirety. Polymerases useful in the invention include any nucleic acid polymerase capable of catalyzing a template-dependent addition of a nucleotide or nucleotide analog to a primer. Any polymerizing enzyme may be used in the invention. A preferred polymerase is Klenow with reduced exonuclease activity. Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W.H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred to as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al., 1998, Proc Natl Acad. Sci. USA 95:14250-->5).

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347 (1975)).

The template/primer complexes are contacted with a nucleotide in the presence of the polymerase under conditions such that the polymerase catalyzes template-dependent addition of the nucleotide to the 3′ terminus of the primer. The nucleotide can be detectably labeled, as described herein, and any incorporated nucleotide is identified by detecting the presence of the label. Unincorporated labeled nucleotides can be removed from the surface prior to detecting the incorporated labeled nucleotide or analog. The process can be repeated one or more times, wherein the template/primer complex(s) are provided with additional nucleotides in the presence of a polymerase, followed by removing the unincorporated labeled nucleotides and detecting the incorporated labeled nucleotides. The sequence of the template is determined by compiling the identified nucleotides. In this manner, the entire sequence of one or more nucleic acids can be determined. In addition, by using single molecule sequencing techniques, determining the sequence for each nucleic acid molecule attached to the surface provides the number of different or unique nucleic acid molecules in the sample. Furthermore, the number of copies of each nucleic acid sequences in a biological sample is also provided.

In order to allow for further extension and detection of subsequently added fluorophore-labeled nucleotides, the fluorophore of the incorporated nucleotide can be destroyed or removed. The fluorophore can be destroyed, for example, photochemical destruction as described in U.S. Pat. No. 6,780,591, the teachings of which are incorporated herein in their entirety. This cycle can be repeated a large number of times if sample losses are avoided. In one embodiment, such losses will be avoided by attaching the nucleic acid molecules or primers to a surface of flow cell and transferring the entire flow cell between a reaction vessel and the fluorescent reader. Alternatively, the nucleotide can be labeled with a fluorphore that is attached to the nucleotide via a cleavable linker as described in Ser. No. 10/866,388 filed Jun. 10, 2004, the teachings of which are incorporated herein by reference in their entirety.

The extension reactions are carried out in buffer solutions which contain the appropriate concentrations of salts, nucleotides and nucleotide polymerizing enzyme required for the enzyme mediated extension to proceed. For guidance regarding such conditions see, for example, Sambrook et al., (1989, Molecular Cloning, A Laboratory Manual, Cold Spring Harbor Press, NY); and Ausubel et al. (1989, Current Protocols in Molecular Biology, Green Publishing Associates and Wiley Interscience, NY).

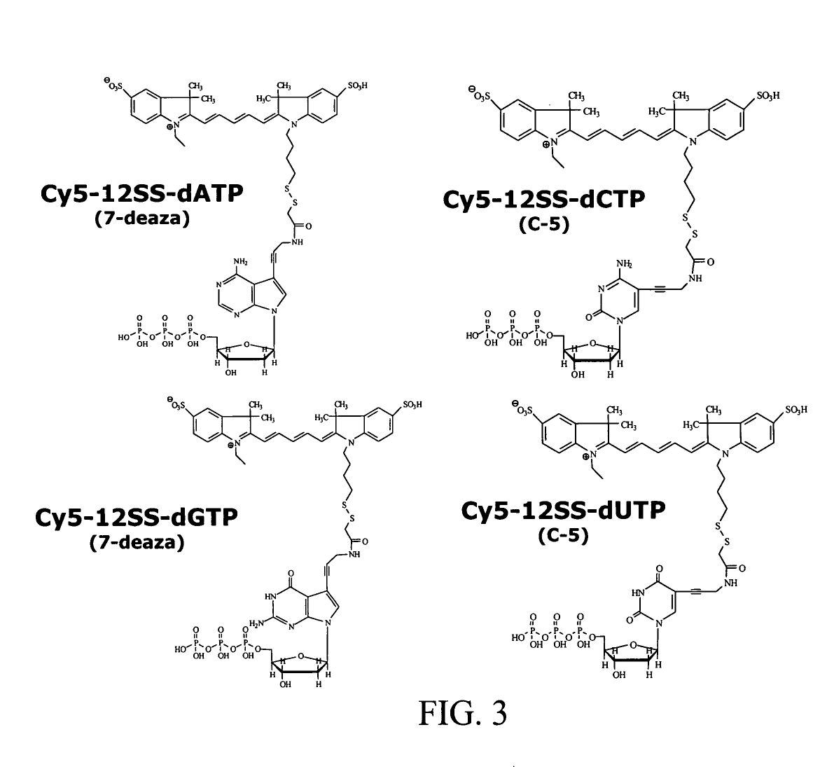

Nucleotides particularly useful in the invention comprise detectable labels. Labeled nucleotides include any nucleotide that has been modified to include a label that is directly or indirectly detectable. Preferred labels include optically-detectable labels, including fluorescent labels or fluorophores. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; di ethyl enetri amine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. FIG. 3 shows the structure of cyanine-5 attached to the four common nucleotides. Labels other than fluorescent labels are contemplated by the invention, including other optically-detectable labels.

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached target nucleic acids.

The present invention provides for detection of molecules from a single nucleotide to a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified COD camera also can be used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. It is sometimes referred to as a high-efficiency photon detection system. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. In other words, the optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached target nucleic acid target molecule/primer complex in the presence of a polymerase. TIR fluorescence microscopy is then used to visualize the attached target nucleic acid target molecule/primer complex and/or the incorporated nucleotides with single molecule resolution.

Measured signals can be analyzed manually or by appropriate computer methods to tabulate results. The substrates and reaction conditions can include appropriate controls for verifying the integrity of hybridization and extension conditions, and for providing standard curves for quantification, if desired. For example, a control nucleic acid can be added to the sample. The absence of the expected extension product is an indication that there is a defect with the sample or assay components requiring correction.

Fluorescence resonance energy transfer (FRET) can be used as a detection scheme. FRET in the context of sequencing is described generally in Braslavasky, et al., Proc. Nat'l Acad. Sci., 100: 3960-3964 (2003), incorporated by reference herein. Essentially, in one embodiment, a donor fluorophore is attached to the primer, polymerase, or template. Nucleotides added for incorporation into the primer comprise an acceptor fluorophore that is activated by the donor when the two are in proximity.

The present invention also includes apparatus suitable for preparation and sequencing of nucleic acids. In one embodiment, the apparatus comprises a flow cell having an inlet, an outlet, and a surface treated to allow nucleic acids to be attached thereto. The apparatus optionally includes nucleic acids or primers attached to the surface. The nucleic acids or primers can be attached to the surface such that at least a portion of the nucleic acids or primers are individually optically resolvable.

The apparatus optionally includes a second surface. One surface can be treated to allow nucleic acids to be attached thereto and the other surface can be treated to allow the immobilization of cells thereto. The two surfaces can be in fluid communication with each other. In one embodiment, the two surfaces are connected by a valve that can be opened to allow fluid communication or closed to prevent fluid communication between the two surfaces.

The apparatus optionally includes a microscope, wherein the flow cell is operably positioned on the microscope stage such that the added nucleotides can be identified using the microscope. In one embodiment, the nucleotides are identified using total internal reflection fluorescence.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for sequencing a nucleic acid, the method comprising the steps of:
a) introducing one or more cells into a flow cell;
b) treating said cells to cause nucleic acids to be released;
c) immobilizing released nucleic acids on a surface of the flow cell; and
d) conducting a sequencing reaction using said immobilized nucleic acids as templates.

2. The method of claim 1, wherein a single cell is introduced into the flow cell.

3. The method of claim 1, further comprising the step of fragmenting said released nucleic acids.

4. The method of claim 1, wherein the cells are lysed and the released nucleic acids are immobilized in separate regions of the flow cell.

5. The method of claim 1, wherein said surface is coated with an epoxide or epoxide derivative.

6. The method of claim 5, wherein said epoxide is derivatized with a member of a binding pair.

7. The method of claim 6, wherein said member of a binding pair is selected from the group consisting of antibodies, antigens, receptors, and ligands.

8. The method of claim 3, wherein the released nucleic acids are fragmented by a method selected from the group consisting of sonication, and enzymatic digestion.

9. The method of claim 1, wherein the immobilized nucleic acids are exposed to primers under conditions suitable for forming a template/primer duplex.

10. The method of claim 9, wherein the primers comprise a homopolymeric nucleotide sequence.

11. The method of claim 9, wherein step d) comprises the steps of:
e) introducing a polymerase and at least one nucleotide species comprising an optically-detectable label under conditions sufficient for template-dependent nucleotide addition to said primer;
f) removing unincorporated nucleotide; and
g) identifying nucleotide species incorporated into said primer.

12. The method of claim 1, wherein the cells are isolated from a biological sample prior to introducing the cells into the flow cell.

13. The method of claim 1, wherein the released nucleic acids are modified with a member of a binding pair and the surface comprises another member of said binding pair, for immobilizing the nucleic acids.

14. A method for sequencing a nucleic acid, the method comprising the steps of:
exposing cells to a flow cell, the flow cell comprising an inlet and an outlet;
extracting nucleic acids from said cells;
attaching said nucleic acids to a surface associated with said flow cell, such that at least a portion of said nucleic acids are individually optically resolvable;
hybridizing a primer to said nucleic acids to form template/primer duplexes;
exposing said duplexes to optically-labeled nucleotides and polymerase under conditions that allow template-dependent nucleotide addition to said primer; and
identifying nucleotides added to said primer.

15. The method of claim 14, wherein about 1000 cells are exposed to the flow cell.

16. The method of claim 14, further comprising the step of fragmenting said nucleic acids prior to attachment to said surface.

17. The method of claim 14, further comprising the step of adding a predetermined number of nucleotides to the 3′ end of said nucleic acids.

18. The method of claim 17, wherein said primer is complementary to said predetermined number of nucleotides.

19. The method of claim 14, wherein said nucleic acids are directly attached to said surface.

20. The method of claim 14, wherein said primers are attached directly to said surface.

21. The method of claim 14, wherein both said nucleic acids and said primers are attached directly to said surface.

22. The method of claim 14, wherein said nucleic acids are attached to said surface via a member of a binding pair.

23. The method of claim 22, wherein said member of a binding pair is selected from the group consisting of an antibody, and antigen, a receptor, and a ligand.

24. The method of claim 14, wherein said surface is an epoxide-coated surface.

25. The method of claim 24, wherein said epoxide coating is passivated to prevent non-specific binding.

26. The method of claim 25, wherein said surface is passivated with phosphate.

27. The method of claim 14, wherein said surface is glass or fused silica.

28. The method of claim 14, wherein said exposing and detecting steps are repeated at least once.

29. The method of claim 14, wherein the flow cell is operably positioned on a microscope stage such that the added nucleotides can be identified using the microscope.

30. The method of claim 29, wherein the nucleotides are identified using total internal reflection fluorescence.

31. The method of claim 14, wherein said optically labeled nucleotides are labeled with a fluorescent dye selected from the group consisting of fluorescein, rhodamine, cyanine, Cy5, Cy3, BODIPY, alexa, and derivatives thereof.

32. An apparatus comprising a flow cell having an inlet and an outlet, and a surface treated to allow attachment of nucleic acids thereto.

33. The apparatus of claim 33, wherein said surface comprises an epoxide or epoxide derivative.

34. The apparatus of claim 32, wherein said epoxide is derivatized with a member of a binding pair.

35. The apparatus of claim 32, wherein said binding pair is selected from the group consisting of biotin/streptavidin and antibody/antigen.

36. The apparatus of claim 32 further comprising nucleic acids or primers attached to said surface such that at least a portion of said nucleic acids or primers are individually optically resolvable.

37. The apparatus of claim 33, further comprising a microscope, wherein the flow cell can be operably positioned on a microscope stage such that the added nucleotides can be identified using the microscope.

38. The apparatus of claim 35, wherein the nucleotides can be identified using total internal reflection fluorescence.
